(19) United States
(12) Patent Application Publication
(10) Pub. No.: US 2009/0198287 A1
(43) Pub. Date: Aug. 6, 2009

(54) BONE FIXATION DEVICE AND METHOD OF USE THEREOF

(76) Inventor: Mark Hsien Nien Chiu, Toronto (CA)

Correspondence Address: BERESKIN AND PARR LLP/S.E.N.C.R.L., s.r.l., 40 KING STREET WEST, BOX 401, TORONTO, ON, M5H 3Y2, CA

(21) Appl. No.: 12/025,198

(22) Filed: Feb. 4, 2008

(51) Int. Cl. A61B 17/56; A61B 19/00

(52) U.S. Cl. 606/301; 128/898

(57) ABSTRACT

A device for flexibly connecting bones, and a method of using the device for repairing a syndesmotic joint of an ankle are disclosed. The connector has, spaced sequentially, a first portion, a central portion and a second portion, and the portions define a longitudinally extending axis. The method comprises securing the first portion of the connector to a tibia of a patient proximate the syndesmotic joint, securing the second portion of the connector to a fibula of the patient proximate the syndesmotic joint, and, positioning at least a portion of the central portion having increased flexibility in the syndesmotic space.

### FIELD OF THE INVENTION

The invention relates to a method and apparatus for fixation of bones. More specifically the invention relates to a device for flexibly connecting bones, and a method of using the device for repairing the syndesmotic joint of an ankle.

### BACKGROUND OF THE INVENTION

The ankle syndesmosis (S) is the joint between the distal tibia (T) and the distal fibula (F), where the tibia and fibula are held together by connective ligaments (L), as shown in FIG. 1. At the ankle syndesmosis, the tibia and the fibula are supported and held together by interosseous membrane and three main ligaments: the anterior inferior tibiofibular ligament, the posterior inferior tibiofibular ligament, and the transverse ligament. During normal gait, the fibula moves with respect to the tibia. For example, referring to FIG. 1, body weight and muscle contraction during walking actively pulls the fibula (F) downward relative to the tibia (T), in a direction indicated by arrow A₁. Additionally, during the phase of walking at which ankle dorsiflexion occurs, the fibula rotates in a plane transverse to the direction of arrow A₁ with respect to the tibia, in a direction indicated by arrow A₂, which is external rotation. The amount that the fibula moves is variable, however some studies indicate that the fibula may move in direction A₁ by about 1-2 mm and in direction A₂ by about 2 degrees.

A common type of ankle injury is injury to the ankle syndesmosis. This type of injury is sometimes called a high ankle sprain. In an ankle syndesmosis injury, at least one of the ligaments connecting the bottom ends of the tibia and fibula bones is torn. Disruption is caused by external rotation forces on the foot and ankle. Progressive disruption of these ligaments results in increased diastasis and instability Mild cases of ankle syndesmosis injury without instability and widening of the syndesmotic space are often treated non-surgically. However, more serious cases may require surgery. Typical surgery to repair the ankle syndesmosis involves placement of one or more stainless steel or bioabsorbable screws through the fibula (F), across the syndesmotic space (S), and into the tibia (T).

### SUMMARY OF THE INVENTION

In one broad aspect, a method for repairing a syndesmotic joint of an ankle is provided. The method involves using a connector comprising a unitary nitinol body. The connector has, spaced sequentially, a first portion, a central portion and a second portion, and the portions define a longitudinally extending axis. The method comprises securing the first portion of the connector to a tibia of a patient proximate the syndesmotic joint, securing the second portion of the connector to a fibula of the patient proximate the syndesmotic joint, and positioning at least a portion of the central portion in the syndesmotic space.

Embodiments in accordance with this broad aspect may be advantageous because the central portion may be substantially flexible, and may therefore bend, flex, or deflect to accommodate the movements of the joint. Accordingly, the connector may permit more natural range of movement for the joint then current surgical methods, and may promote healing of the joint. Additionally, the connector may have a substantially high fatigue resistance, and may therefore be used in the body for an extended period of time without breaking. Furthermore, the connector may prevent bone shielding which leads to osteopenia. Additionally, the connector may be substantially corrosion resistant.

In some embodiments the method further comprises passing the first portion through one of the fibula and the tibia, through the syndesmotic space and into the other of the fibula and the tibia, and positioning the second portion in the one of the fibula and tibia that does not contain the first portion.

In some embodiments, each of the first portion and the second portion comprise screw threads on an outer surface thereof, and the method further comprises screwing the first portion into the tibia and screwing the second portion into the fibula.

In some embodiments, the method further comprises selecting the central portion to have a flexibility to secure the tibia and fibula in a spaced apart relationship on opposed sides of the syndesmotic space such that when normal physiologic forces are applied to the syndesmotic joint, the tibia moves with respect to the fibula by at least 40% of an uninjured syndesmotic joint.

In some embodiments, the method comprises selecting the central portion to have a modulus of elasticity of 20-80 GPa. In further embodiments, the method comprises selecting the central portion to have a modulus of elasticity of 30-40 GPa.

In some embodiments, the method further comprises selecting the central portion to have an external surface having an absence of screw threads.

In some embodiments, the method further comprises selecting a connector such that the central portion is configured to deflect from the longitudinal axis when a force is applied transverse to the longitudinal axis while the first and second portions remain secured in position and to re-align with the longitudinal axis when the force is removed, and to undergo at least 400,000 cycles of deflection and re-alignment without breaking.

In some embodiments, the method further comprises positioning the first end adjacent a cortex of the tibia at a region opposed to the fibula.

In some embodiments, the connector is positioned such that a portion of the central portion is embedded within at least one of the tibia and the fibula. In further embodiments, the connector is positioned such that a portion of the central portion is embedded within the fibula and another portion of the central portion is embedded within the tibia.

In another broad aspect, a bone fixation device is provided. The bone fixation device comprises a longitudinally extending nitinol body having a longitudinally extending axis. The bone fixation device further comprises a first portion comprising an outer surface having at least one bone engagement member securable to a first bone, and a second portion comprising an outer surface having at least one bone engagement member securable to a second bone. A central portion extends between the first portion and the second portion.

In some embodiments, the nitinol comprises from 50% to 60% nickel, and from 40% to 50% titanium by weight.

In some embodiments, the central portion has a length of from 5 to 20 mm. In some further embodiments, the first bone is the tibia and the first portion has a length of from 20 to 50 mm and the second bone is the fibula and the second portion has a length of from 5 to 20 mm.

In some embodiments, the first portion defines a first outer diameter, the second portion defines a second outer diameter, and the central portion defines a third outer diameter less than the first and second outer diameters.

In some embodiments, the central portion has an absence of screw threads.

In some embodiments, the central portion is configured to deflect from the longitudinal axis to allow the second bone to move relative to the first bone in a direction transverse to the longitudinal axis by a distance comparable to a natural movement of the first bone relative to the second bone. In some embodiments, the first bone is a tibia of a patient and the second bone is a fibula of the patient and the distance is comparable to the movement of the tibia relative to the fibula at the syndesmotic joint.

In some embodiments, the bone fixation device has a cycle fatigue of at least 400,000 cycles.

In some embodiments, the bone engagement members comprise screw threads. In some further embodiments, the bone fixation comprises a screw, preferably wherein the central portion has an absence of screw threads.

In some embodiments, the bone fixation device comprises a head adjacent the second portion comprising a slot for receiving a tool. In further embodiments, the first end comprises an additional slot for receiving a tool.

In some embodiments, the bone fixation device extends linearly.

In another broad aspect, a bone fixation device is provided that comprises a longitudinally extending body comprising a first portion comprising an outer surface having at least one bone engagement member securable to a first bone; a second portion comprising an outer surface having at least one bone engagement member securable to a second bone; and, a central portion extending between the first portion and the second portion, the central portion being fabricated from a material having a modulus of elasticity of between 20 GPa and 80 GPa.

In some embodiments, the bone fixation device has a rigidity that is sufficient to secure the first bone and the second bone in a normal anatomical position during normal movement of a portion of a body containing the first bone and the second bone.

In some embodiments, the first bone is a tibia, the second bone is a fibula, and the normal movement is a swing phase of walking.

In some embodiments, the bone fixation device is a screw, and at least one of the bone engagement members comprises a screw thread.

In some embodiments, a diameter of the central portion is at least 2 mm.

### BRIEF DESCRIPTION OF THE DRAWINGS

These and other advantages of the present invention will be more fully and particularly understood in connection with the following description of the preferred embodiments of the invention in which:

FIG. 1 is a front plan view of a syndesmotic joint of a human;

FIG. 2 is a front plan view of an embodiment of a bone fixation device of the present invention;

FIG. 3 is a front plan view of an alternate embodiment of a bone fixation device of the present invention;

FIG. 4A is a front plan view of an embodiment of a bone fixation device of the present invention positioned in a syndesmotic joint of a human, showing the tibia and fibula in a rest position;

FIG. 4B is a front plan view of an embodiment of a bone fixation device of the present invention positioned in a syndesmotic joint of a human, showing the fibula vertically displaced from the tibia;

FIG. 5A is a top plan view of an embodiment of a bone fixation device of the present invention positioned in a syndesmotic joint of a human, showing the tibia and fibula in a rest position; and

FIG. 5B is a top plan view of an embodiment of a bone fixation device of the present invention positioned in a syndesmotic joint of a human, showing the fibula rotationally displaced from the tibia;

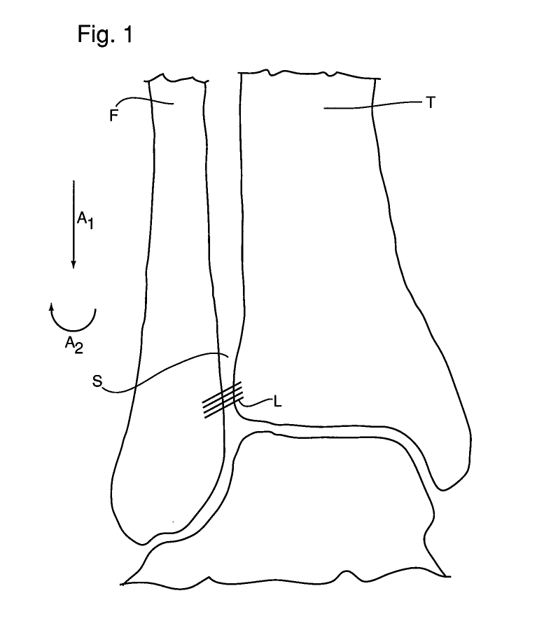

### DETAILED DESCRIPTION OF THE INVENTION

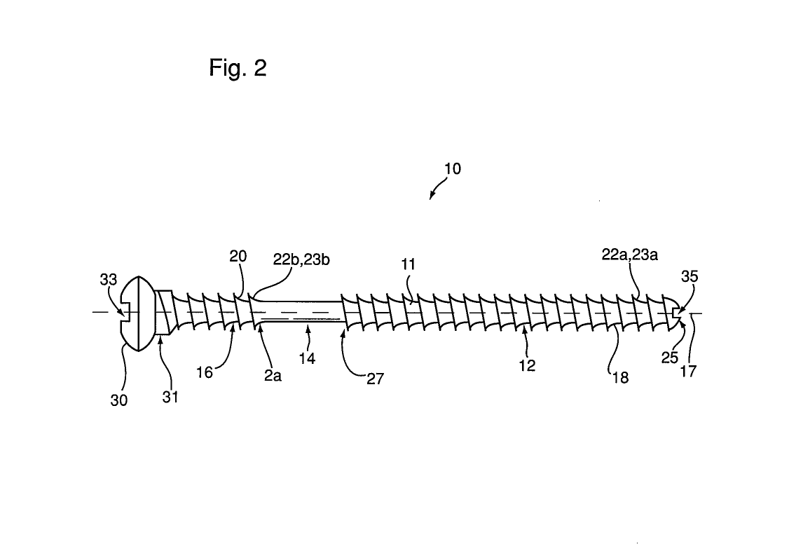

Referring to FIG. 2, an embodiment of a bone fixation device 10 of the present invention is shown. Bone fixation device 10 is a connector comprising a unitary body 11, and is configured to repair injuries, such as ankle syndesmosis injuries, by flexibly connecting two bones, such as the tibia and the fibula. Bone fixation device 10 has a longitudinally extending axis 17, and comprises a first portion 12, a central portion 14, and a second portion 16. Central portion 14 extends along axis 17 between first portion 12 and second portion 16. Bone fixation device 10 is configured such that in use, first portion 12 is secured to a first bone, second portion 16 is secured to a second bone, and at least a portion of central portion 14 extends between the bones.

In order to secure each of first portion 12 and second portion 16 to first and second bones, each of first portion 12 and second portion 16 preferably have an outer surface 18, 20 comprising at least one bone engagement member 22a, 22b. By providing each of first 12 and second 16 portions with a bone engagement member, rather than only first portion 12, over compression or over tightening of the bones may be minimized or prevented.

In the embodiment shown in FIG. 2, bone engagement members 22a, 22b comprise screw threads 23a, 23b, which extend outwardly from outer surfaces 18, 20. Accordingly, bone fixation device 10 is secured to first and second bones, by screwing first portion 12 through a first bone and then into second bone, and screwing second portion 16 into the first bone, such that central portion 14 is positioned at least partially between first bone and second bone.

Screw threads 23a, 23b, may be of a variety of configurations. In the embodiment shown, screw threads 23a extend along first portion 12 from a position adjacent a first end 25 of first portion 12 to a position adjacent a second end 27 of first portion 12. Screw threads 23b extend along second portion 16 from a position adjacent a first end 29 of second portion 16 to a position adjacent a second end 31 of second portion 12. In alternate embodiments, screw threads 23a, 23b may extend along only a portion of each of first portion 12 and second portion 16. In the preferred embodiment, the screw threads are configured to have a diameter and thread pitch compatible with existing orthopedic systems, such that existing tools may be used. Accordingly, in some embodiments, the pitch of screw threads 23a, 23b is between 0.5 mm and 2.5 mm. In the preferred embodiment, screw threads 23a, 23b have a pitch of about 1.25 mm. Furthermore, the screw threads are preferably buttress shaped.

In alternate embodiments, bone engagement members 22a, 22b may be another member other than a screw thread, such as one or more hooks, barbs, or pins. For example, as shown in FIG. 2, each of first portion 12 and second portion 16 may comprise one or more barbs 28 extending outwardly from outer surface 18 of first portion 12 and outer surface 20 of second portion 16, and being angled in a direction towards optional head 30. Barbs 28 may be configured such that when bone fixation device is inserted into first and second bones, for example using a hammer, barbs 28 provide minimal resistance to oppose the insertion; however, if force was applied in a direction indicated by arrow A to bone fixation device 10 to remove it from the first and second bones, barbs 28 would engage the bones and resist the motion. In yet alternate embodiments, connector 10 may be a dowel, and bone engagement members may comprise the outer surfaces 18, 20 of first 12 and second 16 portions of connector 10, which may be sized to frictionally engage the bones. Preferably, as exemplified, bone fixation device 10 is a screw.

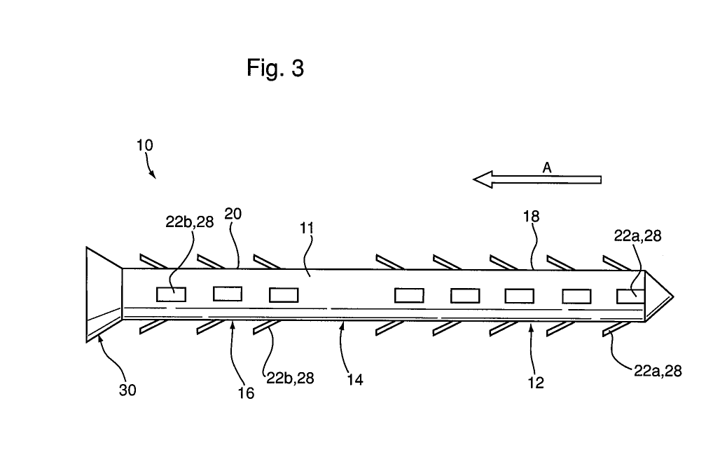

In any of the above-described embodiments, bone fixation device 10 may further comprise a head 30 adjacent second portion 16. In some embodiments, head 30 may comprise a slot 33 for receiving a screwdriver or a drill bit. Alternatively, head 30 may be substantially flat, for striking with a hammer (see for example FIG. 3).

In any of the embodiments, first end 25 of first portion 12 may comprise a slot 35 for receiving a tool such as a screwdriver or a drill bit. Such a slot may be useful if connector 10 breaks while in use in the body. In such cases, connector 10 may be removed from the body by unscrewing second portion 16 from the fibula and by unscrewing first portion 12 longitudinally through and out of the tibia

As previously mentioned, central portion 14 extends between first portion 12 and second portion 16. Central portion 14 is configured to deflect from the longitudinal axis to allow the second bone to move relative to the first bone in a direction transverse to the longitudinal axis by a distance that is preferably comparable to a natural movement of the first bone relative to the second bone. Central portion 14 may be of a variety of shapes, and is preferably cylindrical. In order to provide a central portion 14 that has the requisite flexibility, central portion 14 may have a reduced diameter, be made of a more flexible material, and/or be constructed to have a reduced number of, and preferably an absence of, elements that increase the structural rigidity of central portion 14, such as by having an absence of screw threads.

In the embodiment exemplified in FIGS. 2 and 3, central portion 14 is provided with an absence of screw threads, barbs, or other bone engagement members. That is, the outer surface of central portion 14 is substantially smooth. The provision of screw threads on central portion 14 provides elements that increase the rigidity of the central portion. In alternate embodiments, central portion may be provided with a textured outer surface. For example, the outer surface of central portion 14 may be provided with screw threads.

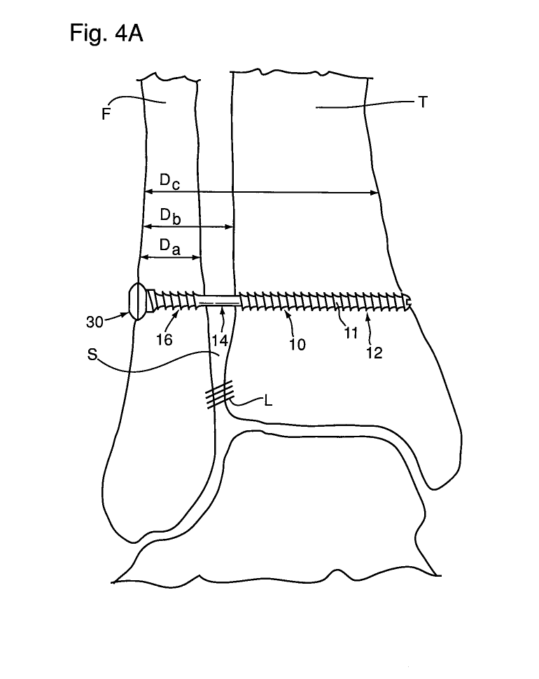

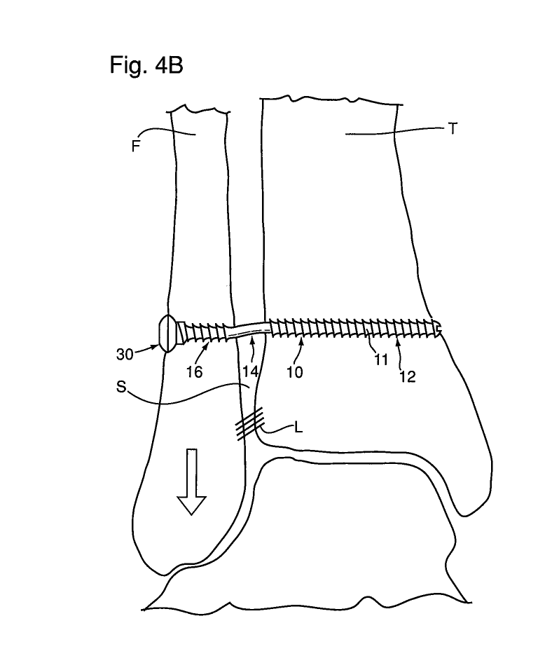

Bone fixation device 10 may be sized depending on the intended use of bone fixation device 10. In some embodiments, as shown in FIG. 4A, bone fixation device 10 is preferably used to repair a syndesmotic joint. It will be appreciated that the dimensions of bone fixation device 10 may vary depending if bone fixation device 10 is to be used on an adult or a child. In some such embodiments, it may be desired for the length of central portion 14 to be between about 5 mm in length and 20 mm in length and preferably between about 5 mm and about 10 mm in length, in order to span the syndesmotic space (S). Further, it may be desired for central portion 14 to be substantially cylindrical, and to be between about 2 mm and about 3 mm in diameter, in order to be easily positioned in the syndesmotic space (S).

Preferably, first portion 12 and second portion 16 may be sized such that they may be secured to the tibia (T) and the fibula (F), respectively. Accordingly, first portion 12 may have a length of between about 20 mm and about 50 mm, and preferably between about 25 mm and about 35 mm, in order to span the diameter of (i.e. extend through) the tibia (T). Second portion 16 may have a length of about 5 mm to about 20 mm, preferably between about 8 mm and about 13 mm, in order to span the diameter of (i.e. extend through) the fibula (F). First portion 12 and second portion 16 may have an outer diameter of between 2 mm and about 3 mm (not including bone engagement members 22), and with bone engagement members 2 may have an outer diameter of between about 3 mm and about 5 mm.

It will be appreciated that bone engagement members 22a, 22b may be provided on only part of first and second portions 12, 16. Further, it will be appreciated that part of central portion 14 may be provided with bone engagement members 22a, 22b. For example, in any embodiment, once inserted into the bones, end 29 may be positioned in the syndesmotic space. Alternately, or in addition, end 27 may be positioned in the syndesmotic space. Since at least part of central portion 14 has, e.g., no screw threads but the same diameter as portion 12, 16 (exclusive of the screw threads), the flexibility of central portion 14 is increased.

Although in the embodiments shown, central portion 14 has substantially the same outer diameter as first and second portions 12, 16 excluding the screw threads on first and second portions (i.e. in the direction transverse to axis 17 between the bases or troughs of the screw threads), in alternate embodiments, central portion 14 may have a diameter that is less then that of first and second portions. Providing central portion 14 with a reduced diameter may serve to increase the flexibility of central portion 14.

As previously mentioned, bone fixation device 10 is configured to flexibly connect a first bone, such as a tibia, and a second bone, such as a fibula, such that if the bones move relative to each other, each of first portion 12 and second portion 16 will remain secured to the bones, and central portion 14 will flex, bend, or deflect to accommodate the movement. More specifically, bone fixation device 10 is configured such that if bone fixation device 10 extends along axis 17, and the first bone moves relative to the second bone, for example in a direction transverse to axis 17, central portion 14 will flex such that bone fixation device deflects from axis 17. Further, bone fixation device 10 may be configured such that if the first bone returns to its original position, central portion 14 will realign with axis 17.

In order to provide central portion 14 with the ability to repeatedly flex, bend, or deflect, at least central portion 14, and preferably all of body 11 of bone fixation device 10 is fabricated from a superelastic or shape memory material such as nitinol (nickel titanium alloy), which has a low modulus of elasticity (preferably from about 20 GPa to about 80 GPa), and a high fatigue resistance (preferably at least 400,000 cycles without failure) Preferably all of body 11 is made of nitinol, which allows connector 10 to flex, bend, or deflect to provide a relatively natural range of movement to joints.

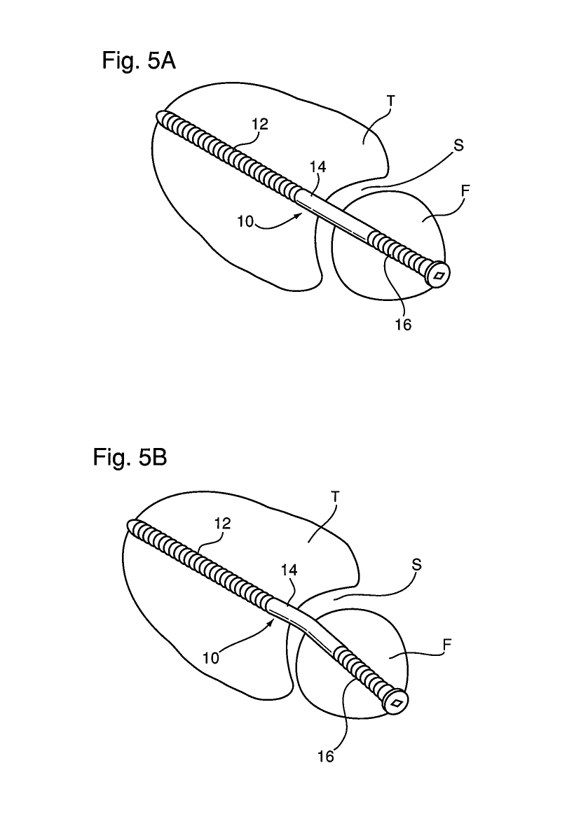

For example, in some embodiments, connector 10 is used to connect the tibia and the fibula to repair the syndesmotic joint of an ankle. As previously mentioned, during normal walking in a human patient, the fibula moves downward (vertically) with respect to the tibia, and externally rotates with respect to the fibula. As shown in FIG. 4A and 5A, during the swing phase of walking where the foot is in mid-air, and minimal forces are applied to the joint and the joint is at rest, central portion 14 of connector 10 may be in a substantially straight or linear configuration. During the stance or pushing off phase of walking, when forces are applied to the joint and the fibula moves downward (vertically) and externally rotates with respect to the tibia, central portion 14 may deflect both downwardly, as shown in FIG. 4B, and rotationally, as shown in FIG. 5B, in order to accommodate the movement and allow the joint to approximate to its natural range of movement. When the swing phase of walking is again resumed, and minimal forces are applied to the joint, the central portion may again return to a straight configuration. The amount of movement which connector 10 allows will depend on the particular configuration of and material used to make connector 10, as well as the nature of the joint being repaired. That is, factors such as scar formation, prolonged immobilization, or severe associated leg injuries may reduce syndesmotic motion. However, in some embodiments, when normal physiologic forces are applied to the joint, connector 10 may allow the tibia to move with respect to the fibula by a distance that is at least 40%, and in some cases 100%, of the distance allowed in the uninjured syndesmotic joint, and by an angle that is at least 40%, and in some cases 100% of the angle allowed in the uninjured syndesmotic joint. In some particular embodiments, connector 10 may allow the fibula to rotate by between about 1 degree and about 4 degrees, and to move downwardly by between about 0.5 mm and 3 mm, and more specifically between about 1 mm and about 2 mm during walking.

In addition to allowing connector 10 to flex, bend, or deflect, the use of nitinol allows connector 10 to be used in the body for an extended period of time, without breaking or otherwise failing. For example, some embodiments of connector 10 may be able to undergo up to 400,000 or more cycles, and preferably more than 1,000,000 cycles, of deflection and re-alignment, without failing. Additionally, due to the low modulus of elasticity of nitinol, the use of connector 10 may reduce bone shielding which leads to osteopenia. Furthermore, the use of nitinol may minimize or prevent corrosion of connector 10 in the body.

The particular alloy of nitinol used for connector 10 may vary depending on the particular application. In some embodiments, bone fixation device is fabricated from a nitinol comprising between 50% to 60% nickel, and from 40% to 50% titanium by weight. It will be appreciated that any formulation of nitinol known now or in the future maybe used. For example, in some embodiments, the nitinol may comprise additional components other than nickel and titanium, such as one or more standard or known additives. In the preferred embodiment, bone fixation device is fabricated from a nitinol comprising about 45 wt % nickel and about 55 wt % titanium. The nitinol is preferably in the martensitic form at body temperature (37° C.), but may be in an austenitic form. The transformation temperature may be above or below 37° C., and is preferably approximately 60° C. In some embodiments, the nitinol may be configured to have a modulus of elasticity of between 20 GPa and 80 GPa. In the preferred embodiment, the nitinol has a modulus of elasticity of between about 30 GPa and 40 GPa. Furthermore, in some embodiments, surface processing treatments such as mirror polishing or oxide coating may be applied to connector 10 to further reduce corrosive forces.

As previously mentioned, the entirety of connector 10 may be made from nitinol, or only central portion 14 may be made from nitinol. For example, first and second portions may be made from stainless steel, and may be affixed to central portion 14, for example by welding.

Bone fixation device 10 may have a variety of uses in the body. In some particular embodiments, bone fixation device 10 may be used to repair damaged joints, such as joints that have been sprained. In one particular embodiment, as exemplified herein bone fixation device 10 may be used to repair an ankle syndesmosis, as will presently be described. Other potential uses may include repair of the coracoacromial joint, acromioclavicular joint, and symphysis pubis joints which involve ligaments which allow normal motion between attached bones. It will be appreciated that in any such uses, bone fixation device 10 has a rigidity that is sufficient to secure the first bone and the second bone in a normal anatomical position during normal movement of a portion of a body containing the first bone and the second bone. For example, if bone fixation device 10 is used to repair an ankle syndesmosis, then the bone fixation device has sufficient rigidity to secure the tibia and fibula in position during normal movement of the leg (e.g., the swing phase of walking). The required rigidity will vary depending upon the bones that are to be secured together.

Prior to positioning bone fixation device 10 in the ankle syndesmosis, a variety of optional steps may be performed. In a first step, the ankle syndesmosis may be positioned into its normal configuration by pressing the tibia and fibula together at or just above the level of the ankle. A drill may then be used to create a hole through the fibula and into the tibia. The distance that the hole extends into the tibia may vary depending various factors, for example the type and severity of injury. However, in the preferred embodiment, the hole is drilled through the entirety of the tibia, from the medial tibial cortex to the lateral tibial cortex. The hole may be created through the widest part of the fibula, approximately 2-4 cm above the ankle joint line. In some embodiments, a 2.5 mm drill bit may be used to create the hole. The hole may be created in an anteromedial direction towards the tibia, and parallel to the joint line. A depth gauge may then be used to measure the distance D_{A} from the lateral fibular cortex to the medial fibular cortex, the distance D_{B} from the lateral fibular cortex to the medial tibial cortex, and the distance D_{C} from the lateral fibular cortex to the lateral tibial cortex (shown in FIG. 4A). The distance across the syndesmotic space D_{S} may be approximated by the difference between distance D_{B} and distance D_{A}. An appropriately sized bone fixation device 10 may then be selected based on these measurements. For example, if distance D_{A} is 10 mm, distance D_{B} is 20 mm, and distance D_{C} is 50 mm, a bone fixation device 10 that has a first portion 12 length of 10 mm, a second portion 16 length of 30 mm and a central portion 14 length of 10 mm may be selected.

When the joint has been prepared, bone fixation device 10 may be used to repair the joint by securing first portion 12 to the tibia, securing second portion 16 to the fibula, and positioning at least a portion of central portion 14 in the syndesmotic space. That is, in some embodiments, first portion 12 may be passed through the fibula and into the tibia, central portion 14 may be passed through the fibula and into the syndesmotic space, and second portion 16 may be passed into the fibula. In embodiments wherein bone engagement members 18 comprise screw threads, bone fixation device 10 may be positioned by screwing first portion 12 through the fibula and into the tibia, and screwing second portion 16 into the fibula. In alternate embodiments, bone fixation device 10 may be positioned in another manner, for example by hammering.

Bone fixation device is positioned such that at least a portion of central portion 14 is in the syndesmotic space. In the preferred embodiment, central portion 14 is centered within the syndesmotic space, with a portion extending into and embedded in the tibia, and/or a portion extending into and embedded in the fibula.

In the preferred embodiment, bone fixation device may be positioned such that first portion 12 extends across the entire diameter of the tibia, and end 25 is positioned adjacent the lateral edge of the tibia. Such an embodiment may be advantageous because if bone fixation device 10 breaks while in the body, first portion 12 may be accessed from the medial side of the ankle to remove first portion 12.

After bone fixation device 10 is positioned, and an appropriate healing time has passed, the patient may begin to walk. Due to the ability of central portion 14 to deflect or bend, the patient may be able to walk with greater comfort, and with a more natural gait. Further, the bone fixation device may be able to withstand numerous cycles of walking, without failing. Additionally, due to the low modulus of elasticity of central portion 14, bone shielding which leads to osteopenia may be prevented.

It will be appreciated that certain features of the invention, which are, for clarity, described in the context of separate embodiments or separate aspects, may also be provided in combination in a single embodiment. Conversely, various features of the invention, which are, for brevity, described in the context of a single embodiment or aspect, may also be provided separately or in any suitable sub-combination.

Although the invention has been described in conjunction with specific embodiments thereof, if is evident that many alternatives, modifications and variations will be apparent to those skilled in the art. Accordingly, it is intended to embrace all such alternatives, modifications and variations that fall within the spirit and broad scope of the appended claims. In addition, citation or identification of any reference in this application shall not be construed as an admission that such reference is available as prior art to the present invention.

We claim:

1. A method for repairing a syndesmotic joint of an ankle using a connector comprising a unitary nitinol body and having, spaced sequentially, a first portion, a central portion and a second portion, the portions defining a longitudinally extending axis, the method comprising:
a. securing the first portion of the connector to a tibia of a patient proximate the syndesmotic joint;
b. securing the second portion of the connector to a fibula of the patient proximate the syndesmotic joint; and,
c. positioning at least a portion of the central portion in the syndesmotic space.

2. The method of claim 1, wherein the method further comprises:
a. passing the first portion through one of the fibula and the tibia, through the syndesmotic space and into the other of the fibula and the tibia; and,
b. positioning the second portion in the one of the fibula and tibia that does not contain the first portion.

3. The method of claim 1, wherein each of the first portion and the second portion comprise screw threads on an outer surface thereof, and the method further comprises screwing the first portion into the tibia and screwing the second portion into the fibula.

4. The method of claim 1, further comprising selecting the central portion to have a flexibility to secure the tibia and fibula in a spaced apart relationship on opposed sides of the syndesmotic space such that when normal physiologic forces are applied to the syndesmotic joint, the tibia moves with respect to the fibula by at least 40% of an uninjured syndesmotic joint.

5. The method of claim 1, further comprising selecting the central portion to have a modulus of elasticity of 20-80 GPa.

6. The method of claim 1, further comprising selecting the central portion to have a modulus of elasticity of 30-40 GPa.

7. The method of claim 1, further comprising selecting the central portion to have an external surface having an absence of screw threads.

8. The method of claim 1 further comprising selecting a connector such that the central portion is configured to deflect from the longitudinal axis when a force is applied transverse to the longitudinal axis while the first and second portions remain secured in position and to re-align with the longitudinal axis when the force is removed, and to undergo at least 400,000 cycles of deflection and re-alignment without breaking.

9. The method of claim 1, further comprising positioning the first end adjacent a cortex of the tibia at a region opposed to the fibula.

10. The method of claim 1, wherein the connector is positioned such that a portion of the central portion is embedded within at least one of the tibia and the fibula.

11. The method of claim 1, wherein the connector is positioned such that a portion of the central portion is embedded within the fibula and another portion of the central portion is embedded within the tibia.

12. A bone fixation device comprising a longitudinally extending nitinol body having a longitudinally extending axis and comprising:
a. a first portion comprising an outer surface having at least one bone engagement member securable to a first bone;
b. a second portion comprising an outer surface having at least one bone engagement member securable to a second bone;
c. a central portion extending between the first portion and the second portion

13. The bone fixation device of claim 12, wherein the nitinol comprises from 50% to 60% nickel, and from 40% to 50% titanium by weight.

14. The bone fixation device of claim 12, wherein the central portion has a length of from 5 to 20 mm.

15. The bone fixation device of claim 12, wherein the first bone is the tibia and the first portion has a length of from 20 to 50 mm and the second bone is the fibula and the second portion has a length of from 5 to 20 mm.

16. The bone fixation device of claim 12, wherein the first portion defines a first outer diameter, the second portion defines a second outer diameter, and the central portion defines a third outer diameter less than the first and second outer diameters.

17. The bone fixation device of claim 12, wherein the central portion has an absence of screw threads.

18. The bone fixation device of claim 12, wherein the central portion is configured to deflect from the longitudinal axis to allow the second bone to move relative to the first bone in a direction transverse to the longitudinal axis by a distance comparable to a natural movement of the first bone relative to the second bone.

19. The bone fixation device of claim 12 wherein the bone fixation device has a cycle fatigue of at least 400,000 cycles.

20. The bone fixation device of claim 12 wherein the bone engagement members comprise screw threads.

21. The bone fixation device of claim 12 wherein the bone fixation device comprises a screw.

22. The bone fixation device of claim 12, further comprising a head adjacent the second portion comprising a slot for receiving a tool.

23. The bone fixation device of claim 22, wherein the first end comprises an additional slot for receiving a tool.

24. The bone fixation device of claim 12, wherein the bone fixation device extends linearly.

25. The bone fixation device of claim 18, wherein the first bone is a tibia of a patient and the second bone is a fibula of the patient and the distance is comparable to the movement of the tibia relative to the fibula at the syndesmotic joint.

26. A bone fixation device comprising a longitudinally extending body comprising:
a. a first portion comprising an outer surface having at least one bone engagement member securable to a first bone;
b. a second portion comprising an outer surface having at least one bone engagement member securable to a second bone; and,
c. a central portion extending between the first portion and the second portion, the central portion being fabricated from a material having a modulus of elasticity of between 20 GPa and 80 GPa.

27. The bone fixation device of claim 26, wherein the bone fixation device has a rigidity that is sufficient to secure the first bone and the second bone in a normal anatomical position during normal movement of a portion of a body containing the first bone and the second bone.

28. The bone fixation device of claim 27, wherein the first bone is a tibia, the second bone is a fibula, and the normal movement is a swing phase of walking.

29. The bone fixation device of claim 26, wherein the bone fixation device is a screw, and at least one of the bone engagement members comprises a screw thread.

30. The bone fixation device of claim 26, wherein a diameter of the central portion is at least 2 mm.
